(19) United States
(12) Patent Application Publication
(10) Pub. No.: US 2025/0178880 A1
(43) Pub. Date: Jun. 5, 2025

(54) PUMPLESS DISPENSING

(71) Applicant: Tendo Technologies Inc., Princeton, NJ (US)

(72) Inventors: Marcus Hultmark, Princeton, NJ (US); Yuyang Fan, Princeton, NJ (US)

(73) Assignee: Tendo Technologies Inc., Princeton, NJ (US)

(21) Appl. No.: 18/842,902

(22) PCT Filed: Mar. 2, 2023
(86) PCT No.: PCT/US2023/014347

§ 371 Date: Aug. 30, 2024

Related U.S. Application Data
(60) Provisional application No. 63/315,733, filed on Mar. 2, 2022.

(51) Int. Cl. B67D 1/04; B67D 1/00; B67D 1/08

(52) U.S. Cl. CPC B67D 1/045; B67D 1/0037; B67D 1/0888; B67D 2210/0006

(57) ABSTRACT

A system and method for pumpless delivery of a product are provided. The system generally utilizes one or more containers each containing a fluid, and sensor(s) configured to detect a velocity of the fluid in an output line from the container(s). A controller is configured to control one or more valves using velocity or flow rate data received from the sensor(s) in order to control the flow of fluid(s) from the container(s). A source for generating a pressure differential, generating either a higher pressure or lower pressure than what is in each container, may also be present.

### CROSS-REFERENCE TO RELATED APPLICATIONS

The present application claims priority to U.S. Provisional Patent App. No. 63/315,733, filed Mar. 2, 2022, the entirety of which is incorporated by reference herein.

### BACKGROUND

The present invention relates in general to a system and method for dispensing fluids, such as beverage dispensing or dosing of pharmaceuticals. Present pumpless systems cannot automatically provide accurate dispensing of the desired fluids, resulting in systems that either have significant variance in dispensing, or require a user to control the dispensing of the fluid.

### BRIEF SUMMARY

To provide improved control over the dispensing of a fluid, embodiments of a disclosed system are described herein. In some embodiments, a system for pumpless delivery of a product may include: (1) a container operably connected to the source that contains a fluid to be dispensed, (2) a sensor configured to detect a velocity or flow rate of an output from the container, and (3) a controller configured to control a valve using the velocity or flow rate data received from the sensor.

In some embodiments, the container may be connected to a source for generating a pressure differential to drive the dispensing of the container, such as a pressurized (i.e., non-atmospheric pressure) gas (such as air, CO₂, O₂, N₂, or an inert gas). In some embodiments, this is a high-pressure gas (e.g., above atmospheric pressure). In some embodiments, this is a low-pressure gas (e.g., below atmospheric pressure), that may be generated using, e.g., a venturi device.

In some embodiments, the fluid to be dispensed is a concentrate. In some embodiments, the concentrate may be, e.g., food concentrate, beverage concentrate, flavor concentrate, a pharmaceutical ingredient, a biopharmaceutical ingredient, a therapeutic composition, or an intermediate cosmetic composition (e.g., a cosmetic composition intended to be combined with another fluid stream prior to application to a user's body). In some embodiments, the fluid to be dispensed may be a finished product, such as a finished cosmetic composition or therapeutic finished product. In some embodiments, the finished product may be, e.g., a solution, suspension, or an emulsion.

In some embodiments, the finished product may take various forms, including, e.g., a foam, a lotion, a gel, or a cream. In some embodiments, the pressurized gas causes the fluid to be dispensed without substantially changing its form (e.g., starting with an appropriate fluid in the container as a liquid, it may be dispensed in a way that results in the fluid still being a low-viscosity liquid, while the pressurized gas escapes to, e.g., the atmosphere). In some embodiments, the pressurized gas can be combined in a way that not only causes the fluid to dispense, but also changes its form (e.g., starting with an appropriate fluid in the container as a liquid, it may be dispensed in a way that entraps at least some of the gas in the fluid and causing the fluid is substantially transformed into a foam).

In some embodiments, the pressurized gas is controlled such that a pressure in each container is less than or equal to 100 psig. In some embodiments, the pressurized gas is controlled such that a pressure in each container is less than or equal to 10 psig. In some embodiments, the pressurized gas is controlled such that a pressure in each container is less than or equal to 5 psig. In some embodiments, the pressurized gas is controlled such that a pressure in each container is less than or equal to 2.5 psig. In some embodiments, the pressurized gas is controlled such that the pressure is 1-2.5 psig.

In some embodiments, the container may include a plurality of containers, each operably connected to the source through a manifold. In some embodiments, the manifold may include one or more valves—which may be controlled by the controller—for controlling the flow of the pressurized gas into the containers. In some embodiments, a single valve may be controlled by the controller using sensor data (e.g., voltage, velocity, mass flow rate, or volumetric flow rate).

In some embodiments, a pressure regulator may be positioned between the source and any container, to control the pressure of a flow of the pressurized gas into the container(s). In some embodiments, the regulator is controlled by the controller.

In some embodiments, a pressure sensor is positioned between the source and any container, to measure a pressure of the pressurized gas flowing into the at least one container. In some embodiments, the pressure sensor communicates with the controller.

In some embodiments, a valve may be present between the source and any container, which may be controlled by the controller.

In some embodiments, the system may include a temperature sensor downstream from a container to measure a temperature of an output from the container. In some embodiments, the temperature sensor may communicate with the controller.

In some embodiments, the sensor and controller may be selected and configured to detect flow rates as low as 1 mg/hour. In some embodiments, the sensor and controller may be selected and configured to detect flow rates as low as 1 μL/min. In some embodiments, the sensor and controller may be selected and configured to detect flow rates as low as 100 μL/min. In some embodiments, the sensor and controller may be selected and configured to detect flow rates as low as 1 mL/min. In some embodiments, the sensor and controller may be selected and configured to detect flow rates between 1 mL/min and 10 mL/min. In some embodiments, the sensor and controller may be selected and configured to detect flow rates between 10 mL/min and 1 L/min. In some embodiments, the sensor and controller may be selected and configured to detect flow rates as high as 100 L/min.

In some embodiments, the controller may include a processor. In some embodiments, the processor may control some or all the valves in the system. In some embodiments, the controller comprises a display, which may be a touch-sensitive display. In some embodiments, the controller may include a knob, button, and/or slider for receiving input from a user. In some embodiments, the controller receiving input from a remote user.

In some embodiments, the controller may be configured to (1) cause a fluid to begin to flow from the container past the velocity or flow rate sensor by adjusting a valve, (2) receive data from the sensor as fluid is flowing past, (3) determine a total amount of output from the container based on the received data, and (4) adjust the valve based on the determined total amount of output.

In some embodiments, a method may be provided, which may include receiving voltage data from a sensor as fluid, forced from a container by a pressurized gas, is flowing past the sensor. The method may include determining a velocity, mass flow rate, and/or volumetric flow rate from the container based on the voltage data. In some embodiments the voltage data is current data. The method may include adjusting the valve based on the velocity, mass flow rate, and/or volumetric flow rate.

### BRIEF DESCRIPTION OF DRAWINGS

FIG. 1 is a schematic illustration of one embodiment of a pumpless system.

FIGS. 2A-2E are simplified illustrations of embodiments of containers.

FIG. 3 is an illustration of a velocity sensor used in some embodiments.

FIGS. 4-5 are schematic illustrations of alternate embodiments of pumpless systems.

FIG. 6 is a flowchart of an embodiment of a method.

FIGS. 7-8 are schematic illustrations of alternate embodiments of a pumpless system.

### DETAILED DESCRIPTION

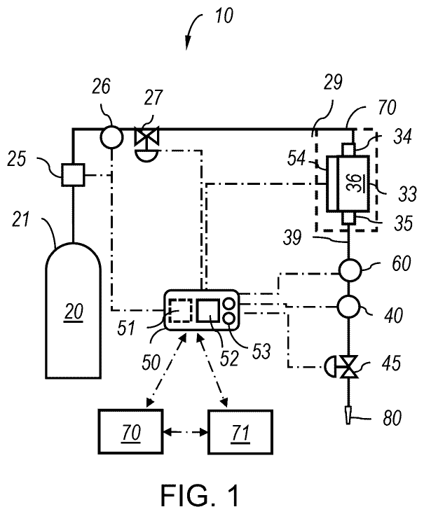

Referring to FIG. 1, a system 10 for pumpless delivery of a fluid can be seen. The system generally comprises a source 21 for a pressurized gas 20, where the source is operably coupled to at least one container 33 that contains a fluid 36 to be dispensed.

The pressurized gas may be any appropriate gas for the fluid to be dispensed. Typically, the gas is expected to be non-chemically reactive with the fluid to be dispensed. Non-limiting examples of the gas include, e.g., air, CO₂, O₂, N₂, or an inert gas (such as He, Ne, or Ar).

In some embodiments, the source 21 is a large or small cylindrical gas container, which may be comprised of a metal. In some embodiments the source of the gas may be a powered device, such as a fan or a compressor. In some embodiments, the source is a gas container according to any one of the specifications under 49 CFR Subpart C (e.g., DOT 3A, 3AX, 3AL, 3E, etc.) or equivalent regulation in non-US countries.

The container may be removably coupled from the rest of the system. For example, in some embodiments, one or more connectors 34, 35 (which may be, e.g., screw fittings) are used to removably couple an input line 29 and output line 39 to the container 33. As will be readily understood, other types of connections, other than screw fittings, are also envisioned. For example, in some embodiments, the components could clamp together. In some embodiments, the container may include a quick-disconnect coupling for connecting to the system.

In some embodiments, the container 33 is a separate component from the one or more connectors 34, 35. In some embodiments, the container and any connectors are a single piece (e.g., molded together as a single component)

The fluid 36 may be any appropriate fluid capable of flowing through the system. In some embodiments, the fluid may be a concentrate, such as a food concentrate, a beverage concentrate, or a flavor concentrate, a scent concentrate, a fertilizer, or may comprise a pharmaceutical ingredient. a biopharmaceutical ingredient. For example, a compounding pharmacy may utilize the system to dispense pharmaceutical ingredients or nutritional ingredients such as total parenteral nutrition.

In some embodiments, the fluid may be a cosmetic or therapeutic concentrate. For example, in some embodiments, an intermediate cosmetic product in one container is co-dispensed with a compatible fragrance and/or colorant composition from a second container, resulting in a finished cosmetic product being dispensed that has the cosmetic intermediate combined with whatever fragrance and/or colorant the user has provided in the second container. In some embodiments, a UV-protective finished product may be formed by dispensed by co-dispensing a base concentrate cosmetic formulation (e.g., which may have no UV-absorbing materials) in one container with different amounts of a UV-protective concentrate in a different container (or one UV-protective concentrate formula selected from one of several different containers, such as one configured to provide an SPF 10 finished product, one configured to provide an SPF 20 finished product, and one configured to provide an SPF 30 finished product) to achieve a particular desired level of UV protection.

In some embodiments, the fluid to be dispensed may be a finished product, such as a beverage, cosmetic or therapeutic product. In some embodiments, the finished product may be, e.g., a solution, suspension, or an emulsion. In some embodiments, the finished product may be in the form of, e.g., a foam, lotion, gel, or cream.

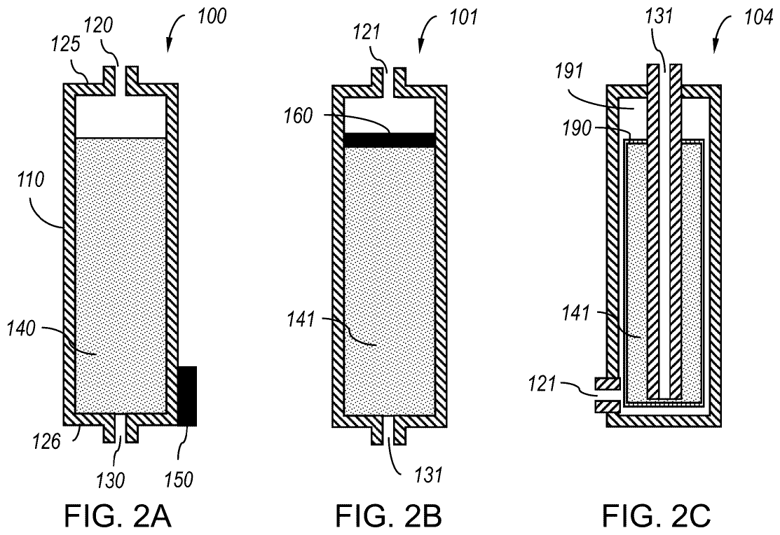

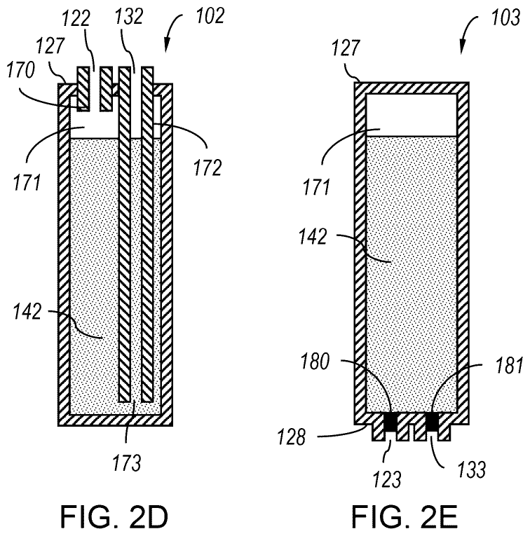

As seen in FIGS. 2A-2D, the design of the container may vary. In some embodiments (see FIG. 2A), the container 100 generally has an exterior wall 110. The exterior wall 110 may be comprised of any appropriate material. In some embodiments, the exterior wall may be rigid. In some embodiments, the exterior wall may be flexible. As used with respect to exterior walls, the term rigid,” is intended to indicate that the walls generally do not lose their overall shape when force is applied. The term “flexible” is intended to indicate walls that can readily bend into retained shapes, or that do not retain a general shape but continuously deform when force is applied.

In some embodiments, the container is comprised of, or consists of, a metal, such as aluminum, or an alloy. In some embodiments, the container is comprised of, or consists of, a polymer, such as HDPE, PET, etc. In some embodiments, the container is comprised of, or consists of, a glass.

The exterior wall will generally define an inlet port 120 and an outlet port 130. In FIG. 2A, the inlet port is on a first end 125 of the container, and the outlet port is on a second end 126, opposite the first end. The pressurized gas is configured to enter into the container via the inlet port 120, and the fluid 140 is then forced out through the outlet port 130. In some embodiments, the inlet port and/or outlet port are configured to couple to the inlet line and/or outlet line respectively via, e.g., threads, barbs or quick-connect fittings.

In some embodiments, circuitry 150 may be present on the exterior of the container. The circuitry 150 may be configured to allow, e.g., circuitry 54 in a housing 70 (see FIG. 1) that is at least partially encompassing the container to communicate with the circuitry 150.

In some embodiments, the circuitry 150 on the container can be used to identify what fluid (or type of fluid) is present in the container.

In some embodiments, the circuitry may include a microchip containing information to be sent to the controller. In some embodiments, the controller can be configured to query the circuitry to determine what is fluid(s) is/are present.

In some embodiments, the circuitry may contain, e.g., resistors, capacitors, etc. In some embodiments, the determination of what material or type of material is present is based on a measured resistance of the circuitry. In such embodiments, the controller could contain (or be operably connected to) a database that correlates measured resistances with fluids or fluid types. For example: 10 ohm=Beverage A, 25 ohm=Beverage B, and 50 Ohm=Beverage C, or 10-100 ohm=Flavor, 100-500 ohm=Beverage, 500-1000 ohm=Food, etc.

Referring to FIG. 2B, in some embodiments, a piston 160 is present in the container to assist with forcing the fluid 141 out of the container 101. The gas will enter into the inlet port 121, push the piston 160 down, forcing fluid out through the outlet port 131. Referring to FIG. 2C, In some embodiments a flexible membrane 190 separates the fluid 141 from the gas 191, the membrane being configured to deform as the fluid is dispensed. In some embodiments the membrane is a bag. In some embodiments, the membrane may comprise a metallized foil bag, and may optionally comprise a polymer. For example, in some embodiments, the bag may include two films, including an aluminum film coupled to a polyethylene terephthalate (PET) film.

Referring to FIG. 2D, in some embodiments, the inlet port 122 and outlet port 132 are positioned on the same end (e.g., end 127) of the container 102. In some embodiments, the inlet port 122 is defined by an inlet tubular portion 170 that extends into the headspace 171 of container, above the fluid 142, while the outlet port is defined by an outlet tubular portion 172 that extends into the fluid 142, such that when gas enters the headspace via the inlet port 122, the increased pressure causes the fluid 142 to enter into an end 173 of the outlet tubular portion 172, and then flow out through the outlet port 132.

In FIG. 2D, the headspace 171 was at the same end (e.g., end 127) of the container 102 where the inlet and outlet ports were located. As seen in FIG. 2E, in some embodiments, in some embodiments, the headspace 171 is at one end (e.g., end 127) of the container 103 (e.g., a “top” end), while the inlet port 123 and outlet port 133 are present at a second end (e.g., end 128) of the container 103, opposite the first end (e.g., a “bottom” end).

In some embodiments, one or more valves may be present in the inlet or outlet ports. In some embodiments, these valves are not controlled by the controller. For example, in some embodiments, valve 180 in the inlet port 123 may be a simple check valve configured to allow a gas to enter but no fluid within the container to exit. In some embodiments, the valve 181 in the outlet port 133 may be, e.g., a fluid dispensing silicone valve, that allows fluid to exit when placed under pressure, but otherwise remain in the container. In some embodiments the valves at the inlet port 123 and outlet port 133 may consist of a foil that is ruptured before use.

Referring back to FIG. 1, the system 10 may also include a sensor 40 for determining a velocity or flow rate of the fluid 36 in the output line 39 from the container 33.

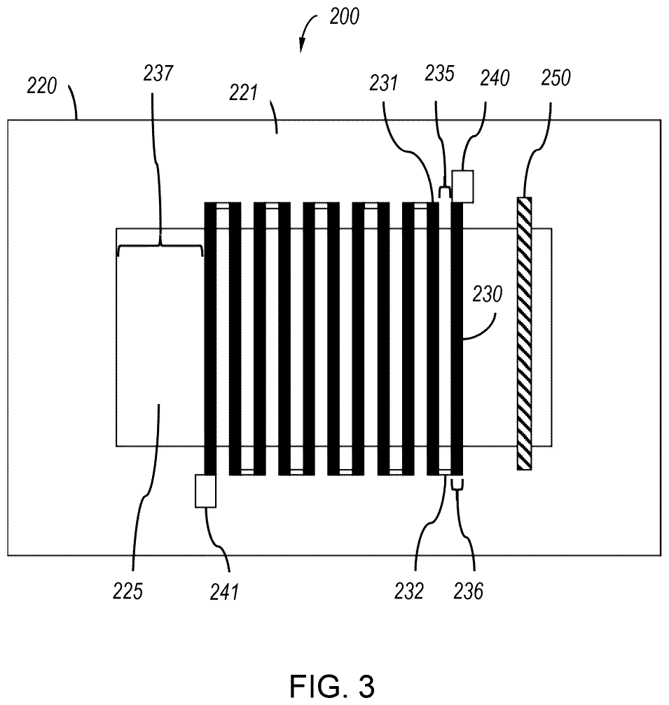

Various velocity or flow rate sensors may be appropriate, based on the fluid 36 and flow conditions in the outlet line 39 of the system. In some embodiments, the velocity sensor is a micro- or nano-scale velocimeter. In some embodiments, the velocity sensor may be, e.g., a multi-wire velocity sensor, such as that seen in FIG. 3. In some embodiments the sensor is an elastic filament velocimeter. In some embodiments the sensor is an impeller based sensor. In some embodiments the sensor is acoustic and/or optical. In FIG. 3, a multi-wire velocity sensor 200 is shown, comprising: a substrate 220 with an outer surface 221 defining an opening 225 through the substrate configured to allow a fluid to flow through the opening in a direction normal to the outer surface and (2) a plurality of wires 230, 231 (which are, e.g., substantially parallel conductive wires) connected 232 in series (such as 5-50 wires, or 10-30 wires), across the opening, in a direction parallel to the outer surface of the substrate, wherein a distance 235 separating each wire from an adjacent wire is between 2 and 10 times the dimension 236 in the transverse direction of the wire. In some embodiments, the distance 237 between one side of the opening 225 and the closest wire to that side is at least 10 times the distance 235 separating each wire from an adjacent wire. In some embodiments, the distance 237 between one side of the opening 225 and the closest wire to that side is as small as the distance 235 separating each wire from an adjacent wire. There may be, e.g., contact pads 240, 241 for allowing current to be applied to the wires. There may be, e.g., additional wire(s) 250 (which may be a single wire, or may be a plurality of wires) that are not connected in series with the plurality of wires 230, 231, where the additional wire(s) 250 have a different sensitivity to temperature as compared to the plurality of wires 230, 231. In some embodiments, the sensor can be an elastic filament velocity sensor.

In various embodiments, the sensor is configured to measure a voltage. That voltage can then be correlated to a velocity, a mass flow rate, or a volumetric flow rate. The correlation may be based on numerous factors, such as thermal conductivity/thermal properties and viscosity. For flow rates, the correlation may also be based on known geometry. The flow rate can be integrated over time to get a total volume (or mass) dispensed. In some embodiments, the correlation is fluid-specific—that is, the system may measure a voltage while it is dispensing a specific fluid, and the system includes a database with a correlation table that was empirically determined using that exact fluid being dispensed. In some embodiments, each fluid may have a defined type or category (which may be based on, e.g., thermal conductivity/thermal properties and/or viscosity of the fluid, or chemical composition), and the correlation may be specific to that type, but not specific to the exact fluid being dispensed. For example, if configured to dispense coffees with optional flavorants, the system may be configured to use a first correlation (of voltage to flow rates) to determine flow rates for any type of coffee (e.g., Columbian dark roast vs. Ecuadoran medium roast, etc.) that the system could dispense, and a second correlation (of voltage to flow rates) for any type of flavorant (e.g., vanilla flavor, caramel flavor, etc.) that could be dispensed.

By measuring flow rates downstream of the fluid containers, the system can avoid issues inherent with conventional systems. For example, in conventional systems, pressure may be measured in an input line to a manifold or container, and then correlate that input pressure to an output flow rate. Thus, such an approach requires pressure must be “stable” to estimate output flow accurately, but even when using a pressure regulator operating at steady state, such input pressures will vary over time, inherently. Further, even if the pressure is known perfectly, variations in the downstream part of the system can affect the actual flow and thus the dispensed volume or mass. Further, when using a manifold coupled to multiple fluid containers, output flow rates from those containers simply cannot be accurately estimated when measuring a single input pressure value (i.e., the pressure to the manifold).

Referring again to FIG. 1, the system 10 may also contain a controller 50 in communication with the sensor 40. In some embodiments, the controller may be configured to control a valve 27 in an input line 29 of the container 33, based on data received from the sensor 40. In some embodiments, the controller may be configured to control a valve 45 in an output line 39 of the container 33, based on data received from the sensor 40. In some embodiments, multiple valves, including valves on the inlet line and/or outlet line are controlled. In some embodiments, the controller may be configured to determine a flow rate based on information from a sensor. Preferably, the determination is not based on a measured pressure; pressure in the disclosed systems will inherently vary over time, and any variation will decrease the accuracy of the flow rate determination.

Further, monitoring a varying pressure (and optionally attempting to control the pressure based on those varying pressure measurements) does not change the underlying problem of varying pressures introducing inaccuracies in pressure measurements. It also does not change the underlying problem with variations in the downstream fluid lines giving resulting in inaccuracies. In certain applications, even very small differences in total flow determinations can be problematic. For example, in pharmaceutical applications, a 5 μg/mL dosage may yield very different results compared to a 10 μg/mL dosage, yet that variation may be inherently present when trying to control a system by monitoring pressure. Further, more preferably a pressure of the fluid leaving one or more containers is not measured;

In some embodiments, the sensor and controller may be selected and configured to detect flow rates as low as 1 mg/hour. In some embodiments, the sensor and controller may be selected and configured to detect flow rates as low as 1 μL/min. In some embodiments, the sensor and controller may be selected and configured to detect flow rates as low as 100 μL/min. In some embodiments, the sensor and controller may be selected and configured to detect flow rates as low as 1 mL/min. In some embodiments, the sensor and controller may be selected and configured to detect flow rates between 1 mL/min and 10 mL/min. In some embodiments, the sensor and controller may be selected and configured to detect flow rates between 10 mL/min and 1 L/min. In some embodiments, the sensor and controller may be selected and configured to detect flow rates as high as 100 L/min.

In some embodiments, the sensor and controller may be selected and configured to detect flow rates between 0.01 mL/min and 100 mL/min.

In some embodiments, the controller may include at least one display 52. In some embodiments, the display may include a touch-sensitive display.

In some embodiments, the controller may include at least one knob, button, or slider 53 for receiving input from a user. For example, in some embodiments, a user may be capable of adjusting the total amount of fluid to be dispensed at a time, the rate at which the fluid is dispensed, or a combination thereof. In some embodiments, the controller is configured to receive input from a remote user (e.g., via wired or wireless communication).

In some embodiments, the controller may operably communicate, wired or wirelessly, with the container 33. As described herein with reference to FIGS. 2A-2D, in some embodiments, circuitry 54 may be present on the exterior of the container. The circuitry 54 may be configured to allow, e.g., a circuitry in housing 70 to communicate with the circuitry 54 on the container to determine or communicate information about the container (e.g., size, fluid, etc.). The housing may partially or completely surround the container.

In some embodiments, the controller adjusts one or more operational parameters (e.g., inlet pressure, target velocity, etc.) based on the fluid in the container.

In some embodiments, the controller includes at least one processor 51. In some embodiments, the at least one processor is configured to control the valve 45.

In some embodiments, the controller, and preferably a processor in the controller, may be configured to cause the fluid 36 to begin to flow from the container 33 past the velocity sensor 40. It will typically do so by controlling one or more valves (e.g., valve 27 and/or valve 45) within the system, to allow the pressurized gas 20 to force the fluid out of the container. As shown in FIG. 1, the velocity or flow rate sensor 40 will typically be downstream from the container 33, but upstream from a valve 45, although embodiments using the opposite arrangement (valve 45 upstream from the velocity or flow sensor 40) are also envisioned. In some embodiments, a valve downstream from the container (e.g., valve 45) is used to control the flow. In some embodiments, a valve upstream from the container (e.g., valve 27) is used to control the flow.

In some embodiments, the controller may be configured to receive data comprising at least one velocity or flow rate from the velocity or flow rate sensor 40. The velocity or flow rate being measured is the velocity or flow rate of the fluid flowing from the container.

In some embodiments, the controller may be configured to determine a total amount of fluid that has passed through the output line from the container based on the received data. In some embodiments, this comprises determining a volumetric flow rate based on the measured velocity and a cross-sectional area of the outlet line 39. In some embodiments, this comprises determining a mass flow rate based on the measured velocity, a cross-sectional area of the outlet line 39, and a density of the fluid 36 in the container. In some embodiments, circuitry (e.g., circuitry 150 in FIG. 2A) on the container communicates the density of the fluid to the controller. In some embodiments, circuitry on the container communicates the composition of the fluid, and the controller accesses a database to determine the composition of the fluid with a viscosity.

In some embodiments, the controller may be configured to adjust one or more valves (e.g., valves 27, 45) based on the determined total output. In some embodiments, the fluid flow rate is stopped after a volume threshold is reached). In some embodiments, the fluid flow rate is slowed after a first volume threshold is reached and stopped when a second volume threshold is reached.

After the controller stops the fluid from flowing, the total amount of output may be reset. For example, if the system or method is filling a series of 500 mL bottles, it may be advantageous for the controller to have a counter that tracks how much is filled into each bottle and reset that counter after 500 mL of fluid is dispensed into each bottle. In such cases, a separate counter may be used to track how many bottles have been filled.

In some embodiments, it may be advantageous for the controller to have a counter that tracks how much fluid has been output in total, or how much remains in the container. For example, if a 1 L container is attached to a system, the container may communicate that the container has 1 L in it. In some embodiments, the controller may use a counter that starts at the defined volume of the container (here, 1 L) and reduces towards 0 as fluid flow is detected by the velocity sensor. In some embodiments, the controller may use a counter that starts at 0 and increases as fluid is detected by the velocity sensor. In some embodiments, the controller may calculate a percentage or amount of fluid remaining in the container (e.g., by dividing the total amount of output by the defined volume of the container). In some embodiments, the removal of a container (or attachment of a new container) may reset the counter.

In some embodiments, the controller may operably communicate, wired or wirelessly, with a remote device 70 (such as a mobile phone, a computer, or remote server). This may be done directly, or indirectly through at least one intermediate device 71 (such as a hub, router, etc.), across one or more networks.

In some embodiments, the controller may communicate a state of the source or the containers to the remote device, which may be configured to display the state to a user. This may include communicating how much fluid is left in a container, or how much gas is estimated to remain in the source. In some embodiments, the controller may communicate operational metrics to a remote device, which may include, e.g., percentage of time spent dispensing a fluid over a period of time (e.g., in the past 8 or 24 hours, since the gas source was last changed, etc.).

Referring to FIG. 1, in some embodiments, the system may contain a pressure regulator 25 and/or a pressure sensor 26 between the source 20 and the container 33. The regulator may be configured to control the pressure of a flow of pressurized gas into the container. In some embodiments, there may be a single regulator between the gas source and the container(s). In some embodiments, there may be a plurality of regulators between the gas source and the container(s). In some embodiments, regulators are connected in series. In some embodiments, regulators are in parallel. The pressure sensor may be configured to measure a pressure of the pressurized gas flowing into the container. In some embodiments, the pressure of the gas leaving the regulator(s) may be 1-2.5 psig.

In some embodiments the velocity or flow rate sensor 40 can be used to detect when the container 33 is empty. In some embodiments, the controller can determine the container is empty if the downstream valve is open but the velocity or flow rate sensor is not providing sensor readings consistent with fluid flowing at an expected rate of flow. In some embodiments, the controller can determine the container is empty if the downstream valve is open, the pressure sensor 26 indicates gas is flowing into the container, and the velocity or flow rate sensor is not detecting the fluid flowing through the system.

In some embodiments the velocity or flow rate sensor 40 can be used to detect when a bubble appears in a liquid line after the container 33. For example, the system may have (e.g., in a database), or may calculate, an expected measured voltage (or range of voltages) for a given fluid being dispensed. If the system measures normal fluid flow, then a voltage outside the expected range for a period of time, then normal fluid flow again, such as if the measured voltage is outside that range for a brief period of time (e.g., less than a second, less than 0.5 seconds, less than 0.1 seconds, etc.), the system may be configured to identify that disturbance as a bubble. In some embodiments, other features of the signal can be used to determine if there is a bubble in the dispensing line, such as amplitude or frequency of the signal. Information may be passed to a controller or remote device that a bubble was detected.

In some embodiments, the system may have identified a voltage, or range of voltages, indicative of one or more gasses (such as the pressurized gas being used as a source). If the sensor detects normal fluid flow, followed by a period where the detected voltage falls in the expected range for the gas, and then the sensor detects normal fluid flow again, the system may be configured to identify that as a bubble.

In some embodiments, the presence of one or more bubbles may indicate a fluid container is low, may indicate an incoming pressure is too high for the fluid being dispensed, or may indicate some form of maintenance is needed. In some embodiments, the system may be configured to provide information to a user (either via a display, error lights, text message, etc.) that a bubble was detected.

In some embodiments, if a fluid other than the expected fluid from the container is detected (e.g., a bubble is detected), the system may be configured to ignore data from the period of time during which the unexpected material was detected when determining total flow through the system. For example, the system is attempting to dispense 8 fluid ounces of a beverage from a single container, and a bubble is detected in the fluid line for 0.1 seconds, it may not make sense to include data from that 0.1 seconds of time, since the any such data would be related to the bubble, not the beverage.

In some embodiments, the controller is configured to communicate with the sensor and the regulator. In some embodiments, the controller is configured to adjust the various valves and regulators to maintain a pressure within the container 33. In some embodiments, a pressure in each container is less than or equal to 10 psig. In some embodiments, the pressure is less than or equal to 5 psig. In some embodiments, the pressurized gas is controlled such that a pressure in each container is less than or equal to 1.5 psig.

In some embodiments, the system 10 may include at least one temperature sensor 60 downstream of the container (e.g., in or on output line 39). In some embodiments, the temperature sensor 60 is located upstream of the velocity sensor 40. In some embodiments, the temperature sensor 60 is collocated with the velocity sensor 40. In some embodiments, the temperature sensor is between the container 33 and the valve 45 and is configured to measure a temperature of whatever fluid is flowing out of the container. In some embodiments, the temperature sensor is mounted on the container 33. In some embodiments, the temperature sensor 60 may communicate with the controller 50. This temperature may be used for, e.g., determining velocity, volumetric flow rates, and/or mass flow rates or improving the accuracy of sensor 40.

In some embodiments, the system may include a needle 80 (e.g., a fine gauge metal tube) downstream from a valve 45 through which the fluid from the container may flow. In some embodiments, the needle may be a 16-34 gauge needle. In some embodiments, the needle may be a 18-30 gauge needle. In some embodiments, the needle is connected to the valve via a line. In some embodiments, the needle is directly coupled to the valve.

In some embodiments, all containers in the system may be coupled to a single needle.

In some embodiments, the system contains more than one needle but having less needles than containers, where each needle is operably coupled to one or more containers (e.g., if there are N containers, there are M needles where 2≤M≤N−1). For example, in some embodiments, the system may have two or more containers dedicated to providing a carrier fluid (e.g., water) that may be connected to a single needle, and a second additional container dedicated to providing a separate component (such as a flavoring agent) is coupled to its own separate needle.

In some embodiments, each container is coupled to its own needle (e.g., if there are N fluid containers in the system, there are Nneedles).

While the above description relates to a system having a single container, it is readily applicable to systems with multiple containers as well.

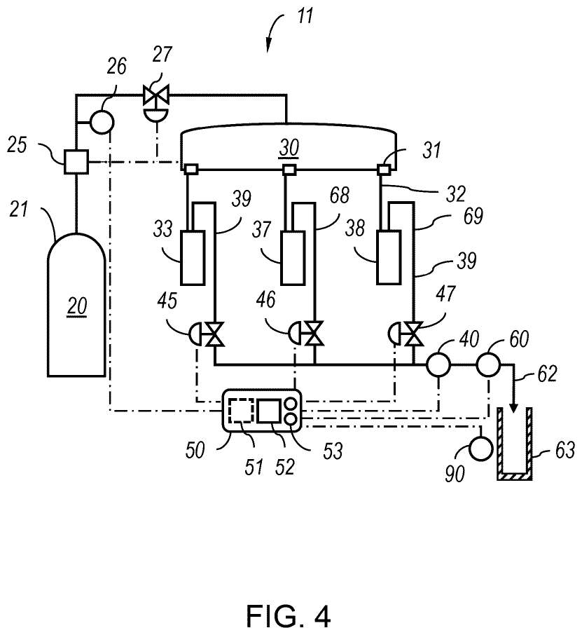

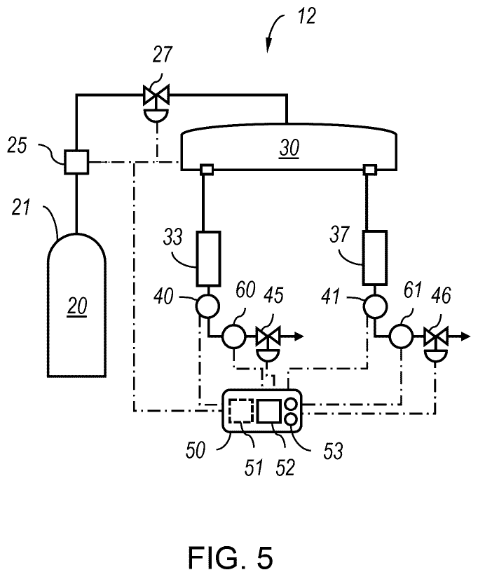

Referring to FIGS. 4 and 5, two different systems are disclosed. In FIG. 4, the system 11 comprises multiple containers 33, 37, 38, each independently filled with a fluid that may (or may not) be the same. In some embodiments, the fluid in each container is the same. In some embodiments, the fluid in each container is different. In some embodiments, one or more fluids in one container (e.g., container 33) are different from one or more fluids in at least one other container (e.g., container 37 or 38).

In some embodiments, the system 11 may include a plurality of containers operably connected to the source 21 through a manifold 30. In some embodiments, the manifold may comprise one or more manifold valves 31 for controlling the flow of the pressurized gas into the one or more containers 33, 37, 38. In some embodiments, the controller is configured to control each of the one or more manifold valves 31.

In some embodiments, each container 33, 37, 39 has a valve 45, 46, 47 associated with its respective output line 39, 68, 69.

In some embodiments, the system is configured such that there is one valve per container controlled by the controller using sensor data. For example, in FIG. 4, this could be valve 27. In this example, the other valves (such as a manifold valve 31) may be controlled based on the desired output product, etc., while other valves (including valve 45, 46, and 47) may be simply turned on or off at appropriate times. In some embodiments the valve is located upstream of the sensor. In some embodiments the valve is located downstream of the sensor. In some embodiments the valves are cycled open and close to effectively reduce the flow rate. This allows one to tune the time it takes to dispense a certain mass or volume.

As seen in FIG. 5, in some embodiments, the system 12 may be configured such that the controller can read the flow of fluid from each container 33, 37 separately, and control the flow of fluid from each container independently. As seen in FIG. 5, one temperature sensor 60, 61, one velocity or flow rate sensor 40, 41, and one valve 45, 46 are downstream from each container 33, 37. Said differently, each container 33, 37 has its own respective temperature sensor, velocity or flow rate sensor, and valve. The controller is capable of reading each sensor independently and controlling each valve independently. In some embodiments, the flow of fluid in one container (e.g., container 33) is controlled based on input only from the temperature sensor 60 and velocity or flow rate sensor 40 downstream from that container. In some embodiments, input from sensors downstream from other containers is included. For example, in some embodiments, the flow of fluid in one container (e.g., container 33) is controlled based on input from the temperature sensor 60 and velocity or flow rate sensor 40 downstream from that container, as well as input from the temperature sensor 61 and velocity or flow rate sensor 41 downstream from at least one other container (e.g., container 37).

In some embodiments, fluid from more than one container can be dispensed simultaneously (e.g., in parallel). In some embodiments, fluid from more than one container can be dispensed serially. In some embodiments (seen in FIG. 4), the fluid from all containers may be combined into a single stream, in a single outlet line 62, prior to dispensing from the system via, e.g, a dispensing nozzle, into an external container 63 (such as a drinking glass or bottle).

In some embodiments, a sensor 90 may be present that detects when an external container is in position to receive fluid from the dispensing nozzle. The sensor may be coupled to the controller. In some embodiments, the system may determine if the external container is correctly positioned prior to dispensing any fluid. In some embodiments, a warning may occur if fluid is dispensed without an external container in position.

In some embodiments (seen in FIG. 5), the fluid from all containers is not so combined prior to dispensing from the system.

Various combinations or permutations of these options are readily envisioned. For example, in embodiments such as those in FIG. 4, where the fluids from each container combine into a single stream, some embodiments may have a single temperature sensor 60 for the combined stream, and multiple velocity or flow rate sensors 40, one downstream from each container prior to being combined, rather than just a single velocity or flow rate sensor for all containers. Similarly, the system may have multiple valves 45 (similar to what is seen in FIG. 5), one downstream from each container, prior to being combined, rather than just a single valve for all containers.

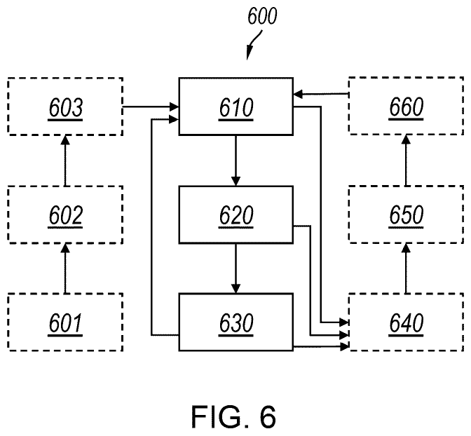

In some embodiments, a method for pumpless dispensing, such as controlling pumpless dispensing, may be provided. Referring to FIG. 6, the method 600 may include receiving 610 voltage data from a sensor as fluid from a fluid container is forced out of the container by a pressurized gas, the fluid flowing from the container and past the sensor.

The method may include determining 620 a velocity, a mass flow rate, or a volumetric flow rate from the container based on the voltage data. In some embodiments, this may include determining a total amount dispensed during a period of time based on the velocity, a mass flow rate, or volumetric flow rate. For example, by integrating one or more determined mass flow rates over time, it is possible to determine a total mass dispensed during a defined period of time.

The method may include adjusting 630 a valve based on the determined velocity, a mass flow rate, or a volumetric flow rate (including values derived from the determined velocity, mass flow rate, or volumetric flow rate) to control the flow of fluid flowing from the container.

In some embodiments, the method may include determining 602 if an external container is positioned to receive the fluid from the fluid container.

In some embodiments, the method may include automatically causing 603 the pressurized gas to force the fluid in the fluid container to begin to flow out of the container and past the sensor, by adjusting a valve (such as the valve adjusted to control the flow of fluid flowing from the container). In some embodiments, this may be based on whether or not an external container is determined to be in position to receive the fluid.

In some embodiments, the method may include providing 601 a system as disclosed herein.

In some embodiments, the method may include communicating 640, over one or more networks, with a remote device. In some embodiments, this communication may include sending information to the remote device. In some embodiments, this information may include information representative of a number of external containers material was dispensed into. In some embodiments, this information may include information representative of an amount of fluid (e.g., mass or volume) dispensed by the system over a period of time. In some embodiments, this information may include information representative of the amount of fluid remaining in one or more fluid container(s). In some embodiments, this information may include information representative of the amount of gas remaining in the source of pressurized gas.

In some embodiments, the method may include communicating 650 with a user, including providing information and/or receiving input from the user. The user may be a user at a remote location, or may be a user nearby the dispensing device. For example, in some embodiments, a display on the dispensing device may display a warning to a nearby user (e.g., fluid in the fluid container may be running low), and may then receive input from the user (e.g., acknowledging and dismissing the warning, or input indicating the dispensing unit should pause or stop dispensing). In some embodiments, this may include displaying information on, e.g., a tablet, laptop, or smartphone a display indicating the current amount of fluid in each container of the dispensing device.

In some embodiments, based on the input from the user, the method may include adjusting 660 (e.g., automatically) a valve to control the flow of fluid flowing from the fluid container. For example, if a user indicates they would like a particular amount of additional fluid to be dispensed, the system should automatically control the flow, using the sensor technique disclosed herein, in order to dispense that amount of fluid. The system may then return to receiving voltage data, making determinations, and adjusting valves.

It will be understood that the optional steps (denoted in FIG. 6 with dashed boxes around them) may, independently, be performed or not as desired, and may be performed in any order. For example, even though communicating 650 with a user is shown as being performed after communicating 640 with a remote device, in some embodiments, communicating with a user may only be performed prior to (or after) the dispensing of fluid into an external container, and no information may be communicated to a remote device.

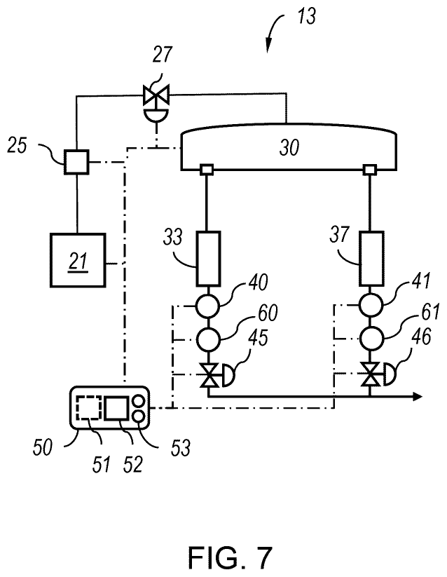

Referring to FIG. 7, in some embodiments, a system 13 may be provided where the source 21 is a device for providing the pressurized gas (such as a fan or compressor). As seen, the source may be operably coupled to the controller 50.

In some embodiments, system may include a separate pressure regulator 25 and/or valve 27 between the source and any containers 33, 37, such as between the source and any manifold 30. In some embodiments, the system may be free of a separate pressure regulator 25 or valve 27 between the source and any containers 33, 37. In some embodiments, only a pressure regulator is between the source and a container. In some embodiments, only a pressure regulator is between the source and a manifold.

In some embodiments, the controller may be configured to adjust a setting for the source (e.g., speed, pressure, etc.) based on a measured/determined flow rate (e.g., via flow rate sensors 40, 41).

In a preferred embodiment, each container has its own flow rate sensor 40, 41. In such embodiments, there is no need to wait for pressure to stabilize, and this avoids any issues of trying to associate a single pressure (or a single flow rate) with multiple cartridges.

In some embodiments, the flow rate sensors in the device can be used for detecting bubbles in the lines. For example, whenever a bubble in the fluid contacts the flow rate sensor, the voltage will typically drop while a gas bubble is in contact with a wire in the sensor. In some embodiments, the controller may be configured to adjust a determined flow rate based on the presence of a bubble. In some embodiments, the controller may be configured to adjust a determined flow rate based on the number of bubbles detected during a period of time. In some embodiments, the controller may be configured to determine a change in voltage (i.e., from a first measurement to a second measurement at a later point in time). In some embodiments, the controller may be configured to determine if the voltage is within a predetermined range (e.g., an expected range of voltages for “normal” operations of the system). In some embodiments, the controller may be configured to determine a bubble is present only if the system is in use (e.g., if the controller has opened one or more valves such that fluid would be expected to flow from the container) and if the voltage is below a predetermined threshold (e.g., a lower limit that may be empirically determined for what would be expected for “normal” use), or a predetermined gas voltage range (e.g., a range of voltages that would be expected for a particular gas to be detected). In some embodiments, the controller may determine a size of an air bubble based on the length of time the voltage is below the predetermined threshold. In some embodiments, the controller may be configured to generate an alert based on the detection of a bubble. In some embodiments, the controller may be configured to generate an alert based on the size of the air bubble detected.

As will be understood, the driving force causing fluid in each container to leave said containers is a pressure differential. In some embodiments, as seen with respect to FIGS. 1, 4, 5, and 7, the driving force is a gas from an upstream source at a pressure greater than the pressure in the ambient pressure, entering the container and displacing the liquid.

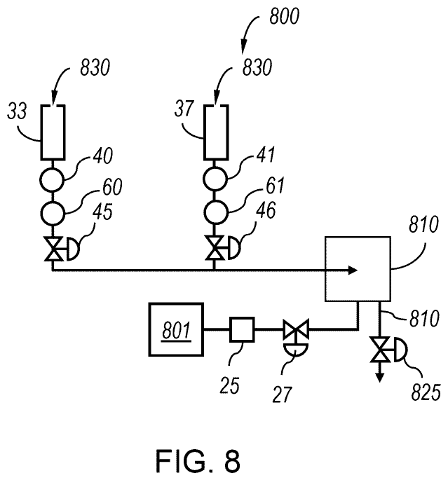

However, as seen in FIG. 8, the system can be reconfigured such that the pressure at the outlet of the container is lower than in the container, yielding a pressure differential which draws the fluid from the container. In FIG. 8, the system 800 generates a low pressure 801, which can be connected to an optional regulator 25 and optional valve 27, and which is operably connected to the container(s) 33, 37, either directly or through an optional output container 810.

In some embodiments, at least one container 33, 37 may have an opening 830 or port for allowing a gas to enter into the container. In some embodiments, all containers containing fluid to be dispensed have such an opening. In some embodiments, the opening is coupled to the atmosphere. In some embodiments, the opening is coupled to a gas source. In some embodiments, the gas source may be used to keep the pressure within the container fixed.

In some embodiments, the output container may be sealed, and may contain additional outputs (e.g., output 820) for allowing the collected fluid and/or the low-pressure gas to leave the container. The outputs may include one or more valves (e.g., valve 825). In some embodiments, the output container may have an opening 835 or port for allowing a gas to enter into the container (e.g., keeping the internal pressure within the containers at ambient/atmospheric pressure).

In some embodiments, the pressure differential is controlled based on the measured flow rates of the fluid(s) leaving the containers. In some embodiments the low pressure 21, is generated by a venturi device (e.g., the container may be operably connected to a device for generating a low-pressure gas, such as a venturi device, etc., as known in the art).

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

Those skilled in the art will recognize or be able to ascertain using no more than routine experimentation many equivalents to the specific embodiments of the invention described herein. Such equivalents are intended to be encompassed by the following claims.

1. A system for pumpless delivery of a product, comprising:
at least one container, each container enclosing an associated fluid, the at least one container operably coupled to a gas;
at least one sensor configured to detect a flow of the associated fluid in an output line from the at least one container; and
a controller in communication with the at least one sensor, the controller configured to control at least one valve using a velocity or a flow rate based on a voltage from the at least one sensor.

2. The system according to claim 1, wherein the at least one container is connected to a source for a pressurized gas.

3. The system according to claim 1, wherein the gas is a low-pressure gas.

4. The system according to claim 3, wherein the low-pressure gas is generated by a venturi device.

5. The system according to claim 1, wherein the associated fluid is a liquid.

6. The system according to claim 1, wherein a pressure in each container is less than or equal to 100 psig.

7-9. (canceled)

10. The system according to claim 2, wherein the at least one container comprises a plurality of containers, each operably connected to the source through a manifold.

11.-34. (canceled)

35. The system according to claim 10, wherein the manifold further comprises one or more valves for controlling the flow of the pressurized gas into the plurality of containers.

36. (canceled)

37. The system according to claim 35, wherein there is at least one valve on at least one of output line from the plurality of containers that is controlled by the controller using data from the at least one sensor.

38. The system according to claim 2, further comprising a pressure regulator between the source and the at least one container, configured to control a pressure of a flow of pressurized gas into the at least one container.

39. (canceled)

40. The system according to claim 2, further comprising a pressure sensor between the source and the at least one container, configured to measure a pressure of the pressurized gas flowing into the at least one container.

41. (canceled)

42. The system according to claim 2, further comprising a valve between the source and the at least one container.

43. (canceled)

44. The system according to claim 1, further comprising at least one temperature sensor collocated with the at least one valve, configured to measure a temperature of a product flowing out of the at least one container.

45. The system according to claim 1, further comprising at least one temperature sensor between the at least one container and external container (63), configured to measure a temperature of a product flowing out of the at least one container.

46. The system according to claim 45, wherein the at least one temperature sensor communicates with the controller.

47. The system according to claim 1, wherein the controller comprises at least one processor.

48. The system according to claim 47, wherein the at least one processor controls the at least one valve.

49-52. (canceled)

53. The system according to claim 1, wherein the product comprises a food concentrate, a beverage concentrate, a flavor concentrate, a pharmaceutical ingredient, a biopharmaceutical ingredient, a fertilizer, or a cosmetic composition.

54-61. (canceled)

62. The system according to claim 1, wherein the controller is configured to:
cause the product to begin to flow from the at least one container, past the at least one sensor;
receive sensor data comprising at least one flow rate from the at least one sensor;
determine a total amount of output from the at least one container based on the sensor data; and
adjust the at least one valve based on the total amount of output.

63. A method for pumpless dispensing, comprising:
receiving voltage data from a sensor as a fluid is flowing past;
determining a velocity, a mass flow rate, or a volumetric flow rate from a container based on the voltage data; and
adjusting a valve based on the velocity, a mass flow rate, or a volumetric flow rate.
